(19) United States
(12) Patent Application Publication
(10) Pub. No.: US 2021/0169501 A1
(43) Pub. Date: Jun. 10, 2021

(54) Bone spur scraping device

(71) Applicant: Xiaogang ZHOU, Nantong (CN)

(72) Inventor: Xiaogang ZHOU, Nantong (CN)

(21) Appl. No.: 16/771,241

(22) PCT Filed: Oct. 23, 2018
(86) PCT No.: PCT/CN2018/111442

§ 371 Date: Jun. 10, 2020

(30) Foreign Application Priority Data
Oct. 22, 2018 (CN) 201821708549.6

(51) Int. Cl. A61B 17/16; A61B 17/3211

(52) U.S. Cl. CPC A61B 17/16; A61B 17/3211

(57) ABSTRACT

The utility model discloses a bone spur scraping device, which includes a bone scraping portion. The bone scraping portion includes a first elongated shell and a blade. One end of the first elongated shell is an open structure, and the One end of the first elongated shell is a closed structure, a limiting slot is opened through both side walls of the first elongated shell, one end of the blade is a tip, and the other end of the blade is Two blades are provided at the flat end, and the flat ends of the two blades are movably inserted inside the first elongated housing, and two blades are provided with a mounting hole near the flat end.

### TECHNOSPHERE

The utility model relates to the technical field of medical instruments, in particular to a bone spur scraping device.

### BACKGROUND TECHNOLOGY

Orthopedics is one of the most common departments in major hospitals. It mainly studies the anatomy, physiology and pathology of the skeletal and muscular system. It uses drugs, surgery and physical methods to maintain and develop the normal shape and function of this system. With the changes of times and society, the orthopedic injury spectrum has changed significantly. For example, diseases such as bone and joint tuberculosis, osteomyelitis, and poliomyelitis have decreased significantly, and the trauma caused by traffic accidents has increased significantly.

Bone spur is a common bone disease in orthopedics. In the prior art, in the process of bone spur surgery, a variety of medical instruments are needed to complete the operation. However, these medical instruments have poor applicability, inconvenient use, poor safety performance, and practicality. The sex is not good. A scraper device is required to scrape the bone spurs on the patient's bones. A polishing device is required when polishing the parts of the patient's bone that are difficult to scrape, especially the position of the patient's joints. Sometimes no suitable polishing device is found It often leads to recurrence of postoperative bone spurs or adhesion symptoms. For this reason, a bone spur scraping device is proposed.

### Contents of the Invention

The purpose of the utility model is to provide a bone spur scraping device, which has good applicability, convenient use, safety and reliability, and good practicability. Part of the polishing, especially the position of the patient's joints, can effectively avoid the recurrence of the patient's postoperative bone spurs or the occurrence of adhesion symptoms to solve the problems raised in the background art.

To achieve the above object, the present invention provides the following technical solution: A bone spur scraping device, including a bone scraping portion, the bone scraping portion includes a first elongated shell and a blade, the first elongated shell One end of the first elongated she is an open structure, and one end of the first elongated shell is a closed structure, the first elongated shell is provided with limiting slots through both side walls, and one end of the blade is a tip, And the other end of the blade is a flat end, the blade is provided with two, and the flat ends of the two blades are both movably inserted inside the first elongated shell, and the two blades are close to the blade A mounting hole is opened at the flat end, and a threaded rod is fixedly installed inside the two mounting holes, and the ends of the two threaded rods away from the blade extend through the limiting groove to the first elongated bar A lock nut is screwed to the outside of both ends of the two threaded rods away from the blade.

By adopting the above technical solution, the blade is used to scrape the bone spurs from the bones of the patient during the operation, and the length of the blade protruding from the first elongated shell can be adjusted, so that the bone scraping portion has good applicability and is easy to use. When the blade is not needed, the blade can be hidden inside the first elongated shell, which can prevent the blade from accidentally injuring the patient or medical staff, so that the bone scraping part has better safety performance. In addition, the blade is provided with two, One of which is used as a spare, can prevent one of the blades from being damaged, and the other blade can continue to be used, so that the bone scraping part is safe and reliable, and will not delay the treatment of the patient. Second, the bone scraping part adjusts the blade to extend the first The length of the elongated shell adopts the operation mode of the lock nut and the threaded rod, which can effectively prevent the accidental sliding phenomenon of the blade during the operation and effectively ensure the smooth and stable operation of the operation.

Preferably, each of the blades is provided with a notch near its tip, the notches are spoon-shaped notches, and the notches are provided with blades.

By adopting the above technical solution, for bone spurs that are inconvenient to scrape on the bones of the patient, the bone spurs can be inserted into the gap, and then the bone spurs can be removed by the blade inside the gap using a push-pull method, thereby making the bone scraping part practical Sex is better.

Preferably, the surface of the first elongated shell is integrally provided with a plurality of antiskid patterns arranged at equal distances.

By adopting the above technical solution, the antiskid pattern can effectively increase the static friction between the surface of the first elongated shell and the hand, which can prevent the occurrence of a gap between the hand and the first elongated shell when the medical personnel operate the instrument Slip phenomenon to avoid accidentally injuring medical staff or patients.

Preferably, it further includes a polishing portion, the polishing portion includes a second elongated housing fixedly mounted on the closed end of the first elongated housing, the second elongated housing away from the first One end of an elongated shell is an open end, and a battery and a mounting seat are fixedly installed side by side inside the second elongated shell, and the side of the mounting seat extending away from the battery extends to the second The outside of the open end of the elongated housing, and an installation slot is opened inside the installation base, an electric motor is fixedly installed inside the installation slot, and a polishing wheel is fixedly installed on the main shaft of the electric motor Located outside the second elongated housing, the electric motor is connected in series with the battery through a wire.

By adopting the above technical solution, an electric motor can be used to drive the polishing wheel to polish the hard-to-scrape parts of the patient's bones, especially the position of the patient's joints, which can effectively avoid the recurrence of postoperative bone spurs or adhesion symptoms.

Preferably, the outer side of the mounting base away from the battery can also be detachably fitted with a protective cover.

By adopting the above technical solution, the protective cover is used to protect the polishing wheel, prevent the polishing wheel from being damaged by collision, and prevent the accidental touch of the control switch during operation of the device, which causes the electric motor to start, thereby preventing the polishing wheel from being injured accidentally Human accidents occur.

Preferably, a control switch and a charging interface are further provided outside the second elongated housing, the control switch is connected in series between the electric motor and the battery, and the charging interface is connected to the The storage batteries are connected in series, and the storage batteries are lithium ion storage batteries.

By adopting the above technical solution, it is convenient to control the operation of the electric motor through the control switch, and it is convenient to charge the battery through the charging interface. In addition, the lithium-ion battery has better battery life and can ensure that the polishing sect ion can work for a longer time.

Compared with the prior art, the beneficial effects of the present invention are

1. In the utility model, the blade is used to scrape the bone spurs from the patient's bones during the operation, and the length of the blade extending out of the first elongated shell can be adjusted, so that the bone scraping portion has good applicability and is easy to use;

2. The utility model, when the blade is not needed, can hide the blade inside the first elongated shell, which can prevent the blade from accidentally injuring the patient or the medical staff, thereby making the bone scraping part safer;

3. In the present utility model, there are two blades, one of which is used as a backup to prevent one blade from being damaged, and the other blade can continue to be used, so that the bone scraping portion is safe and reliable, and will not delay the treatment of the patient;

4. In the utility model, the length of the blade extending from the first elongated shell is adjusted by the operation of the locking nut in conjunction with the threaded rod, which can effectively prevent the blade from sliding accidentally during the operation and effectively guarantee The operation proceeded smoothly and steadily;

5. According to the present invention, for bone spurs that are inconvenient to scrape on the bones of patients, the bone spurs can be inserted into the gap, and then the bone spurs can be removed by the blade inside the gap using a push-pull method, thereby making the bone scraping part practical Better sex;

6. The utility model can use an electric motor to drive the polishing wheel to polish the hard-to-scrape parts of the patient's bones, especially the position of the patient's joints, which can effectively avoid the recurrence of postoperative bone spurs or adhesion symptoms;

7. In this utility model, the protective cover is used to protect the polishing wheel, prevent the polishing wheel from being damaged due to collision, and prevent the control switch from being accidentally touched during the operation of the device, which causes the electric motor to start, thereby preventing the polishing wheel from being injured accidentally. Human accidents are worth promoting and popularizing.

### FIGURE ILLUSTRATION

1 is a schematic structural view of an embodiment of the utility model;

2 is a schematic diagram of an explosion structure of an embodiment of the utility model;

3 is a schematic diagram of an explosion structure of a bone scraping portion of an embodiment of the present invention;

4 is a schematic diagram of an explosion structure of a polishing portion according to an embodiment of the present invention.

In the picture: 1. Bone scraping part; 2. Polishing part; 3. First elongated shell; 4. Limit slot; 5. Blade; 6. Mounting hole; 7. Threaded rod; 8. Lock nut; 9. Notch; 10. Anti-slip pattern; 11. Second elongated shell; 12. Control switch; 13. Battery; 14. Mounting seat; 15. Mounting slot; 16. Electric motor; 17. Polishing wheel; 18. Protective cover.

### Specific Implementation Modalities

The technical solutions in the embodiments of the present invention will be described clearly and completely in conjunction with the drawings in the embodiments of the present invention. Obviously, the described embodiments are only a part of the embodiments of the present invention, but not all of them, example. Based on the embodiments of the present invention, all other embodiments obtained by those of ordinary skill in the art without making creative efforts fall within the protection scope of the present invention.

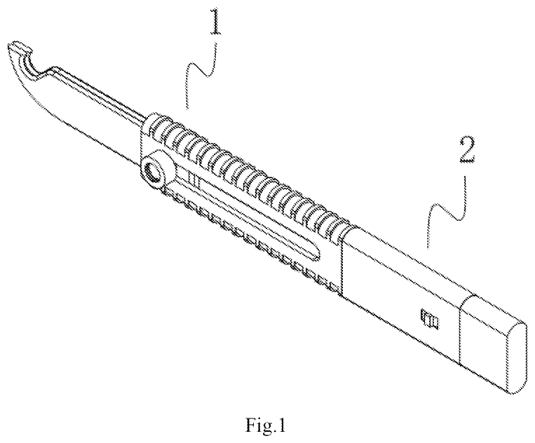

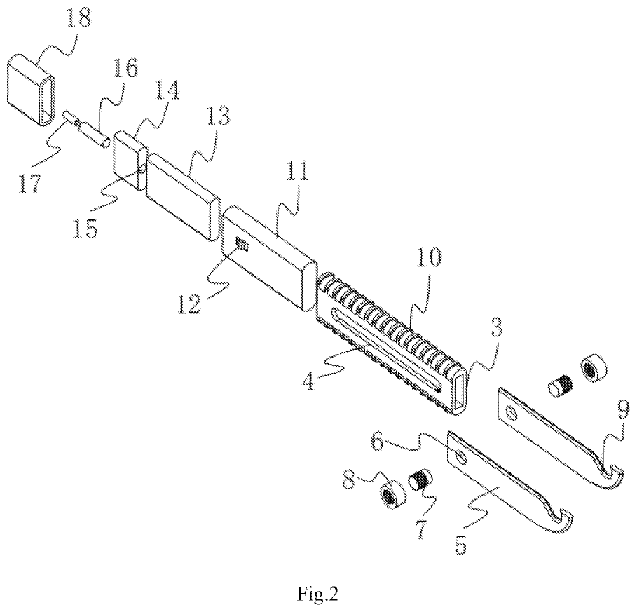

Please refer to FIG. 1-4, the utility model provides a technical solution:

A bone spur scraping device, as shown in FIGS. 1-3, includes a bone scraping portion 1, the bone scraping portion 1 includes a first elongated shell 3 and a blade 5, the first elongated shell 3 One end of the first elongated housing 3 is an open structure, and one end of the first elongated housing 3 is a closed structure. One end is a tip, and the other end of the blade 5 is a flat end, the blade 5 is provided with two, and the flat ends of the two blades 5 are movably inserted inside the first elongated housing 3, Two of the blades 5 are provided with a mounting hole 6 near the flat end thereof. A threaded rod 7 is fixedly installed in the two mounting holes 6 respectively, and both ends of the threaded rod 7 away from the blade 5 pass through A locking nut 8 is screwed to the outside of the two ends of the two threaded rods 7 away from the blade 5 through the limiting groove 4 and extending to the outside of the first elongated housing 3.

The blade 5 is used to scrape the bone spurs from the patient's bones during the operation, and the length of the blade 5 extending out of the first elongated housing 3 can be adjusted, so that the bone scraping portion 1 has good applicability and is easy to use. In addition, When the blade 5 is not needed, the blade 5 can be hidden inside the first elongated housing 3 to prevent the blade 5 from accidentally injuring the patient or medical personnel, thereby making the bone scraping portion 1 have better safety performance. In addition, the blade 5 There are two, one of which is used as a spare to prevent one blade 5 from being damaged, and the other blade 5 can continue to be used, so that the bone scraping portion 1 is safe and reliable, and will not delay the treatment of the patient. Second, the bone scraping Part 1 adjusts the length of the blade 5 extending out of the first elongated housing 3. The operation mode of the lock nut 8 and the threaded rod 7 can effectively prevent the blade 5 from sliding accidentally during the operation and effectively ensure the smooth operation Proceed steadily.

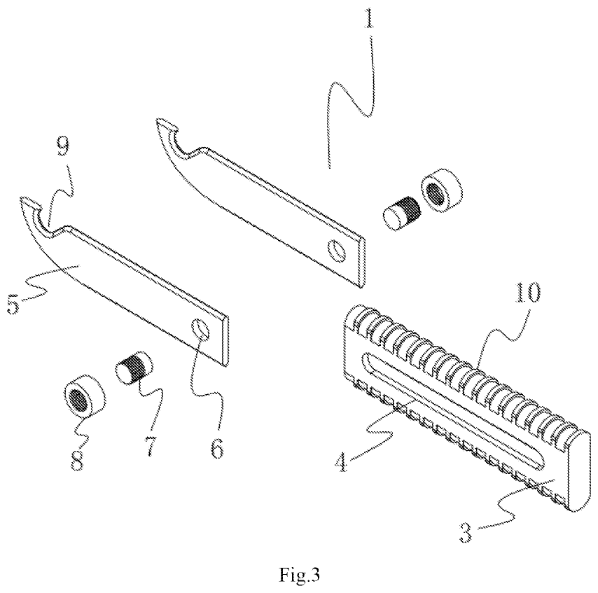

Specifically, as shown in FIG. 3, each of the blades 5 is provided with a notch 9 near its tip, the notches 9 are spoon-shaped notches, and the notches 9 are provided with blades.

For bone spurs that are inconvenient to scrape with the blade 5 on the patient's bones, the bone spurs can be inserted into the notch 9, and then the bone spurs can be removed by the blade inside the notch 9 in a push-pull manner, thereby making the bone scraping portion 1 more practical it is good.

Specifically, as shown in FIG. 3, the surface of the first elongated housing 3 is integrally provided with a plurality of anti-skid patterns 10 arranged at equal distances.

The anti-slip pattern 10 can effectively increase the static friction between the surface of the first elongated housing 3 and the hand, and can prevent slippage between the hand and the first elongated housing 3 when medical personnel operate the instrument. So as to avoid accidentally injuring medical staff or patients.

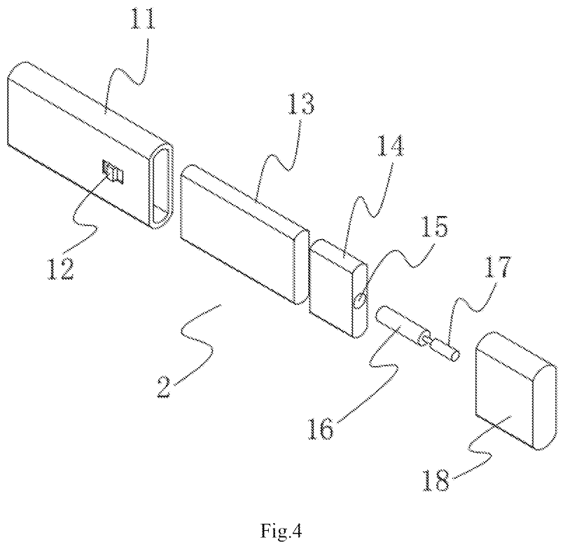

Specifically, as shown in FIGS. 1 and 4, the polishing portion 2 further includes a second elongated housing 11 fixedly mounted on the closed end of the first elongated housing 3, so An end of the second elongated housing 11 away from the first elongated housing 3 is an open end, and a battery 13 and a mounting seat 14 are fixedly installed inside the second elongated housing 11 side by side, The side of the mounting base 14 away from the battery 13 extends to the outside of the open end of the second elongated housing 11, and the inside of the mounting base 14 is provided with a mounting slot 15, and An electric motor 16 is fixedly installed, and a polishing wheel 17 is fixedly installed on the main shaft of the electric motor 16, the polishing wheel 17 is located outside the second elongated housing 11, and the electric motor 16 is connected The battery 13 is connected in series.

The electric motor 16 can be used to drive the polishing wheel 17 to rotate and polish the hard-to-scrape parts of the patient's bones, especially the position of the patient's joints, which can effectively avoid the recurrence of postoperative bone spurs or adhesion symptoms.

Specifically, as shown in FIG. 4, a protective cover 18 may be detachably sleeved on the outside of the side of the mounting base 14 away from the battery 13.

The protective cover 18 is used to protect the polishing wheel 17 and prevent the polishing wheel 17 from being damaged due to collision. At the same time, it can prevent the electric switch 16 from being started by accidentally touching the control switch 12 during operation of the device, thereby preventing the polishing wheel 17 from being injured Human accidents occur.

Specifically, as shown in FIG. 4, a control switch 12 and a charging interface are further provided outside the second elongated housing 11, the control switch 12 is connected in series between the electric motor 16 and the battery 13 In the meantime, the charging interface is connected in series with the battery 13 through a wire, and the battery 13 is a lithium ion battery.

It is convenient to control the operation of the electric motor 16 through the control switch 12 and to charge the storage battery 13 through the charging interface. In addition, the lithium-ion storage battery has better battery life, which can ensure that the polishing section 2 can work for a longer time.

In this embodiment, the electric motor 16 may be a miniature gear motor model ZWBPD024024-6 produced by Shenzhen Zhaowei Electromechanical Co., Ltd., which has the advantages of small size and large torque, which meets the requirements of this embodiment.

Working principle: The medical instrument for orthopedics, the blade 5 is used to scrape the bone spurs from the patient's bones during the operation, and the length of the blade 5 protruding from the first elongated housing 3 is adjustable, thereby making the bone scraping portion 1 applicable Good, and easy to use, in addition, when the blade 5 is not needed, the blade 5 can be hidden inside the first elongated housing 3, which can prevent the blade 5 from accidentally injuring the patient or medical staff, thereby making the bone scraping portion The safety performance is better. In addition, there are two blades 5, one of which is used as a backup to prevent one blade 5 from being damaged, and the other blade 5 can continue to be used, so that the bone scraping portion 1 is safe and reliable, and will not delay the The treatment of the patient, secondly, the length of the blade 5 adjusting the blade 5 extending out of the first elongated housing 3 adopts the operation mode of the lock nut 8 and the threaded rod 7 to effectively prevent the blade 5 from accident during the operation The sliding phenomenon occurs, which effectively guarantees the smooth and stable operation of the operation; the electric motor 16 can drive the polishing wheel 17 to polish the hard-to-scrape part of the patient's bone, especially the position of the patient's joint, which can effectively avoid the patient's postoperative bone spurs. Relapse or adhesion symptoms.

How to use: During use, sterilize the entire instrument before use, and then loosen the lock nut 8 on one side, then push the blade 5 out, and push the lock nut 8 to the proper position. Hold the first elongated housing 3 to scrape the bone spurs on the patient's bones. For the bone spurs on the patient's bones that are inconvenient to scrape with the blade 5, insert the bone spurs into the gap 9 and then use the push-pull method The bone spurs are removed by the blade inside the notch 9; the protective cover 18 is pulled out, the electric motor 16 is turned on by the control switch 12, and the polishing wheel 17 is driven by the electric motor 16 to rotate to polish the hard-to-scrape part of the patient's bone.

Installation Method:

In the first step, first install the bone scraping portion 1, insert the flat end of the blade 5 into the inside of the first elongated housing 3, then fix one end of the threaded rod 7 into the inside of the mounting hole 6, and then lock the nut 8 can be screwed on the outside of the threaded rod 7;

The second step is to install the polishing section 2. The second elongated housing 11 and the first elongated housing 3 can be connected in an integrated manner, and then the battery 13 is fixedly installed on the second elongated housing 11 Inside, the electric motor 16 is fixedly installed in the mounting groove 15 inside the mounting seat 14, and then the control switch 12 and the charging interface are installed and wired, and then the mounting seat 14 is fixedly installed inside the second elongated housing 11, The polishing wheel 17 is fixedly installed on the main shaft of the electric motor 16, and the protective cover 18 is installed.

Although the embodiments of the present invention have been shown and described, those of ordinary ski 11 in the art can understand that various changes and modifications can be made to these embodiments without departing from the principle and spirit of the present invention, Replacement and modification, the scope of the present invention is defined by the appended claims and their equivalents.

1. A bone spur scraping device, characterized in that It includes a bone scraping portion (1), the bone scraping portion (1) includes a first strip-shaped housing (3) and a blade (5), the first strip One end of the shaped shell (3) is an open structure, and one end of the first elongated shell (3) is a closed structure. The first elongated she (3) is opened through both side walls A limiting slot (4), one end of the blade (5) is a tip, and the other end of the blade (5) is a flat end, the blade (5) is provided with two, and two of the blades (5)), The flat ends are movably inserted inside the first elongated housing (3), and two blades (5) are provided with a mounting hole (6) near the flat end and two mounting holes (6) A threaded rod (7) is fixedly installed inside, and both ends of the threaded rod (7) away from the blade (5) extend through the limiting groove (4) to the first length A lock nut (8) is screwed to the outside of the bar-shaped housing (3), and the two ends of the threaded rods (7) away from the blade (5) are screwed.

2. A bone spur scraping device according to claim 1, characterized in that each blade (5) is provided with a notch (9) near its tip, and the notches (9) are spoon-shaped notches, Moreover, the notches (9) are provided with blades.

3. A bone spur scraping device according to claim 1, characterized in that the surface of the first elongated housing (3) is integrally provided with a plurality of anti-skid patterns (10) arranged at equal distances.

4. The bone spur scraping device according to claim 1, characterized in that it further comprises a Polishing part (2), wherein the polishing part (2) includes a fixedly installed on the first elongated casing (3) and is closed A second elongated shell (11) on the end, an end of the second elongated shell (11) away from the first elongated shell (3) is an open end, and the second A battery (13) and a mount ng seat (14) are fixedly installed inside the elongated housing (11) side by side, and the mounting seat (14) extends from the side away from the battery (13) to the second elongated bar An outer side of the open end of the shaped shell (11), and an installation slot (15) is opened inside the mounting seat (14), and an electric motor (16) is fixedly installed inside the installation slot (15) (16) A polishing wheel (17) is fixedly mounted on the main shaft, the polishing wheel (17) is located outside the second elongated housing (11), and the electric motor (16) is connected to the The storage batteries (13) are connected in series.

5. A bone spur scraping device according to claim 4, characterized in that a protective cover (18) can be detachably sleeved on the outside of the side of the mounting seat (14) away from the battery (13).

6. A bone spur scraping device according to claim 4, characterized in that: a control switch (12) and a charging interface are further provided outside the second elongated housing (11), and the control switch (12) Connected in series between the electric motor (16) and the battery (13), the charging interface is connected in series with the battery (13) through a wire, and the battery (13) is a lithium ion battery.
